(19) United States
(12) Patent Application Publication
(10) Pub. No.: US 2002/0039578 A1
(43) Pub. Date: Apr. 4, 2002

(54) Methods for treating disease with antibodies to CXCR3

(76) Inventors: Subhashini Arimilli, Fremont, CA (US); Walter Ferlin, Antibes (FR); Shrikant Deshpande, Fremont, CA (US); Simonetta Mocci, Belmont, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/818,705

(22) Filed: Mar. 27, 2001

Related U.S. Application Data
(63) Continuation-in-part of application No. 09/754,941, filed on Jan. 4, 2001.
Application No. 09/754,941 is a continuation of application No. 09/164,186, filed on Sep. 30, 1998, now Pat. No. 6,171,590.

(51) Int. Cl. A61K 39/395

(52) U.S. Cl. 424/143.1; 424/185.1

(57) ABSTRACT

The present invention provides antibodies directed against the CXCR3 chemokine receptor protein for use in compositions and methods for the treatment, and prevention of inflammatory diseases and conditions.

### CROSS-REFERENCES TO RELATED APPLICATIONS

This application is a continuation-in-part of U.S. patent application Ser. No. 09/754941, filed Jan. 4, 2001, which is a continuation of U.S. patent application Ser. No. 09/164,186, filed on Sep. 30, 1998, which issued as U.S. Pat. No. 6,171,590 on Jan. 9, 2001. This application also claims priority to U.S. patent application Ser. No. 09/535,598 filed on Mar. 27, 2000.

### BACKGROUND OF THE INVENTION

The present invention relates to novel compositions and methods for inhibiting inflammatory responses associated with autoimmune diseases. In particular, it relates to antibodies directed against an extracellular domain CXCR3 receptor polypeptide.

Chemokines constitute a family of small molecular weight cytokines that are produced in inflammation and regulate leukocyte recruitment. These molecules are ligands for seven transmembrane G protein linked receptors that induce a signaling cascade costimulation for T cell activation in addition to participating in transendothelial migration of leukocytes (Oppenheim et al. Ann. Rev. Immunol. 9:617-648 (1991), Premback et al. Nat. Med. 2:1174-1178 (1996)). Two subfamilies of chemokines, referred to as CC and CXC, have been discovered. CC and CXC chemokines are distinct from each other in their N terminal amino acid sequence which starts either with cysteine-cysteine or cysteine-X-cysteine where X is typically another L-amino acid. They are also distinct in their binding pattern to their receptors. For example, the CC chemokines bind to CC receptors and not to CXC receptors and vice versa.

Different chemokines regulate the trafficking of distinct populations of hemopoietic cells by activating specific 7-transmembrane receptors expressed by these cells (Baggiolini et al. Adv. Immunol. 55:97-179 (1994); Gerard et al. Curr. Opin. Immunol.6:140-145 (1994)). Recent publications indicate that the Th1 and Th2 subsets of regulatory T cells are uniquely characterized by the chemokine receptors CXCR3 and CCR3, respectively (Sallusto, et al. J. Exp. Med. 187:875-883 (1998); Bonecchi, et al J. Exp. Med. 187:129-134 (1998); Qin, et al. J. Clin Invest. 101:746-754 (1998)). Several studies have correlated the expression of three specific chemokines, IP-10, RANTES, and MCP-1, produced by astrocytes in the CNS with the presence of inflammatory infiltration within this tissue during the early phase of EAE (Ransohoff, et al. FASEB J. 7:592-600 (1993); Glabinski et al. Am. J. Pathol. 150:617-630 (1995) Godiska, et al. J. Neuroimmunol. 58:167-176 (1995); and Eng et al. Neurchem. Res. 21:511-525 (1996)). While all three chemokines have been shown to be capable of recruiting T lymphocytes in certain experimental models, IP-10 has been demonstrated to be specific for this lineage of hemopoietic cells (Taub et al. J. Exp. Med. 177:1809-1814 (1993)); Carr, et al. Proc. Natl. Acad. Sci. USA 91:3652-3656 (1994); and Farber, J. Leukoc. Biol. 61:246-257 (1997). MBP-immunized rats intrathecaly infused with an antisense phosphorothioate oligonucleotide to crg-2 (the murine homologue of human IP-10) show reduced disease clinical score of EAE (Wojcik, et al. J. Pharmacol. Exp. Ther. 278:404-410 (1996)). In addition, higher expression of some of the chemokine receptors such as CXCR3 on IL2 activated human T lymphocytes and not on resting T lymphocytes has been demonstrated (Loetscher et al. J. Exp. Med. 184:963-969 (1996)).

Multiple sclerosis (MS) is a T cell-dependent autoimmune disease caused by localized demyelination in the central nervous system (CNS), with only limited therapeutic options available to patients. Extensive investigation has indicated that these autoreactive T lymphocytes frequently, though not always, express the Th1 phenotype of high level production of IFNg, IL-2 and TNFa, with little to no IL-4, IL-5 and IL-10.

Current therapeutics for autoimmune diseases, such as MS, involve the use of antiinflammatory agents or general immunosuppressants. Prior art methods for controlling autoimmune disease fail to provide a simple self-mediated method for specifically eliminating inflammatory responses mediated by chemokines associated with the autoimmune responses. The present invention addresses these and other needs.

### SUMMARY OF THE INVENTION

The present invention provides methods of inhibiting the recruitment of T cells to an inflammation site in a patient. The methods comprise administering to the patient a pharmaceutical composition comprising a pharmaceutically effective amount of an antibody directed against an extracellular domain CXCR3 receptor polypeptide. Typically, the extracellular domain CXCR3 polypeptides are comprised of a polypeptide from the first or fourth CXCR3 extracellular domain.

The extracellular domain CXCR3 polypeptides are preferably conformationally constrained, for example by cyclization. The length of the extracellular domain CXCR3 polypeptide is not critical to the invention. Typically, the peptide consists of from about 10 to about 50 residues, more often between from 15 to about 40 residues. Exemplary extracellular domain CXCR3 polypeptides of the invention include PPCPQDFSLNFDRAFLPA (SEQ ID NO: 3), DILMDLGALARNCGRESRVDVAKS (SEQ ID NO: 7), and MVLEVSDHQVLNDAEVAALLENFSSSYDYGENESDSC (SEQ ID NO: 9).

The extracellular domain CXCR3 polypeptides of the invention are useful for raising antibodies directed against the extracellular domains of CXCR3. Polyclonal and monoclonal antibodies are contemplated by the invention. Preferably, the antibody is monoclonal, more preferably the monoclonal antibody contains human antibody polypeptide sequences, still more preferably the monoclonal antibody consists of fully human antibody sequences.

The antibodies directed against the extracellular domains of CXCR3 can be administered by any of a number of means. Typically they are administered parenterally. In preferred embodiments, the method are used to inhibit recruitment of T cells to inflammation sites in a patient. Typically the inflammatory response is associated with an autoimmune disease, such as multiple sclerosis.

The invention also provides pharmaceutical compositions suitable for use in the above methods.

### BRIEF DESCRIPTION OF THE DRAWINGS

FIG. 1 shows acidification rate changes due to the binding of anti-human CXCR3 Mab or IP-10 to CXCR3-NSO transfectants. 5×10⁵ CXCR3-NSO transfectant cells and untransfected “BONZO-NSO” cells (negative control) were spotted in the microphysiometer chambers along with agarose and 1, 5, 10 mglml anti-CXCR3 (FIG. 1a) or IP-10 (FIG. 1b) was pumped for 10 min. There is a dose dependent increase in the acidification rates with anti-CXCR3 in NSO-CXCR3 transfectant cells. Untransfected BONZO-NSO cells did not show any change in acidification rate changes either with the anti-CXCR3 antibody, or IP-10. The arrow indicates the time that ligands were added to the cells.

FIG. 2 shows the structure of the seven transmembrane G-protein coupled human CXCR3. Only amino acids in the extracellular domains are given (SEQ ID NOS: 1, 2,3,4,5, 6,7).

FIG. 3 is a schematic of the experiments in which peptides vaccines of the invention were used to prevent EAE in mice.

FIG. 4 shows the results of experiments in which peptides vaccines of the invention were used to prevent EAE in mice.

### DEFINITIONS

The term “peptide” is used interchangeably with “oligopeptide” or “polypeptide” in the present specification to designate a series of residues, typically L-amino acids, connected one to the other typically by peptide bonds between the α-amino and carbonyl groups of adjacent amino acids.

The term “cyclic peptide” refers to peptides in which the N-terminal residue is linked to the C-terminal residue either directly or through an intermediate. Examples of links between the two residues include disulfide bonds and thioether linkages as described below.

An “immunogenic chemokine receptor polypeptide” of the present invention is a polypeptide capable of eliciting an immune response against a chemokine receptor molecule associated with inflammation in autoimmune responses in a patient, such as multiple sclerosis. As set forth in more detail below, the sequence of residues in the polypeptide will be identical to or substantially identical to a polypeptide sequence in a chemokine receptor molecule. Thus, a polypeptide of the invention that has a sequence “from a extracellular region of a chemokine receptor molecule” is polypeptide that has a sequence either identical to or substantially identical to the naturally occurring chemokine receptor amino acid sequence of the region.

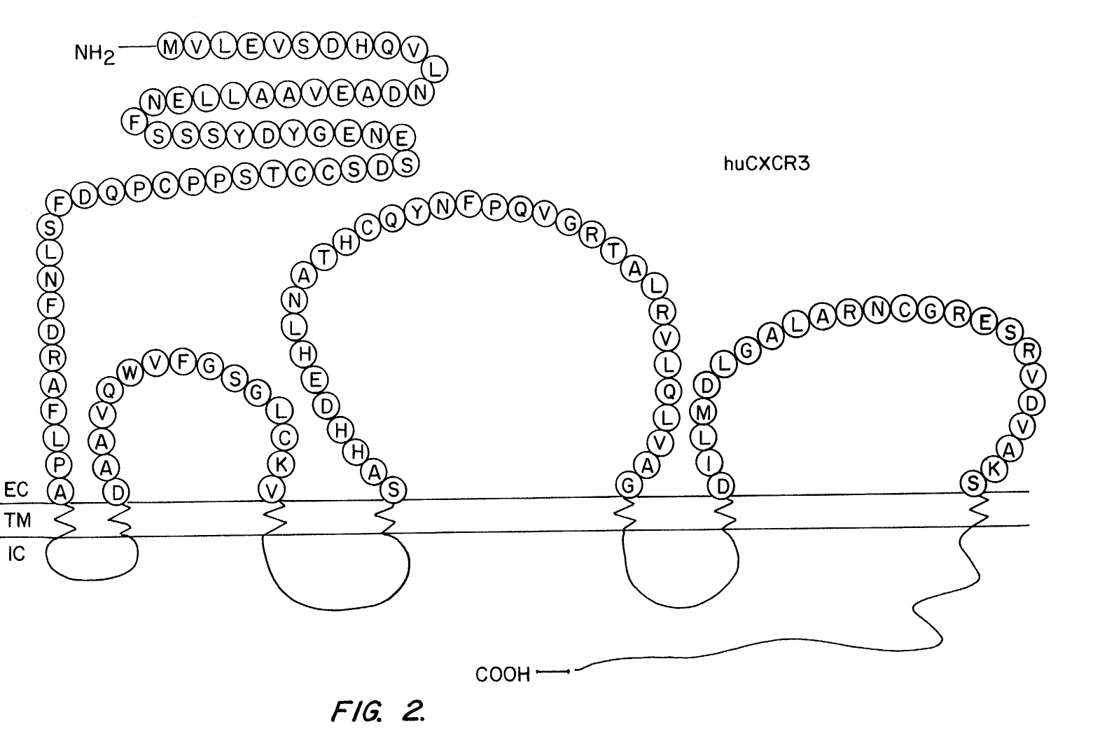

As used herein an “extracellular domain CXCR3 receptor polypeptide” is a region of the molecule, or portion thereof, which is exposed on the surface of a cell expressing the native molecule and has a sequence either identical to or substantially identical to the naturally occurring chemokine receptor amino acid sequence of the region. FIG. 2 provides a schematic of the extracellular domains of the human CXCR3 molecule. This molecule has four extracellular domains designated as SP-1, SP-2, SP-3 and SP-4, starting from the N-terminus. An example of a first and a fourth domain of the extracellular domain of CXCR3 are SEQ ID NO: 10 (MVLEVSDHQVLNDAEVAALLENFSSSYDYGENESDSCCTSPPCPQDFSLNFDRAFL PA) and SEQ ID NO: 7.

As used herein, the term “adjuvant” refers to any substance which, when administered with or before an antigen, increases and/or qualitatively affects the immune response against the antigen in terms of antibody formation and/or the cell-mediated response. Exemplary adjuvants for use in the present invention are provided below.

The phrases “isolated” or “biologically pure” refer to material which is substantially or essentially free from components which normally accompany it as found in its native state. Thus, the chemokine receptor polypeptides of this invention do not contain materials normally associated with their in situ environment, e.g., other surface proteins on T cells. Even where a protein has been isolated to a homogenous or dominant band, there are trace contaminants in the range of 5-10% of native protein which co-purify with the desired protein. Isolated polypeptides of this invention do not contain such endogenous co-purified protein.

The term “residue” refers to an amino acid or amino acid mimetic incorporated in a oligopeptide by an amide bond or amide bond mimetic.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition.

An “antibody” can be derived from sequence of a mammal. A “mammal” is a member of the class Mammalia. Examples of mammals include, without limitation, humans, primates, chimpanzees, rodents, mice, rats, rabbits, sheep, and cows.

“Antibody” also refers to fragments and substitutes for antibodies such as F(ab′)₂, Fab′, and Fab fragments. Additionally the “antibodies” can be single chain antibodies known as ScFv fragments, which are obtained by recombinantly fusing the variable regions of the light and heavy chains of the antigen binding fragment of interest.

An “antibody directed against an extracellular domain CXCR3 receptor polypeptide” is an antibody capable of binding to or recognizing a CXCR3 extracellular region or portion thereof.

### DESCRIPTION OF THE SPECIFIC EMBODIMENTS

The present invention provides antibodies directed against the extracellular domains of CXCR3 for use in compositions and methods for the treatment and prevention of inflammatory responses. The antibodies directed against the extracellular domain peptides of CXCR3 are capable of inhibiting T cell recruitment to an inflammation site. In preferred embodiments the antibodies are directed against extracellular domain peptides of CXCR3 which are conformationally constrained (e.g., cyclized). Administration of antibodies directed against the extracellular domains of CXCR3 results in the specific inhibition of the inflammatory responses mediated by CXCR3 molecules.

The methods can be used to treat any inflammatory response mediated by a CXCR3 molecule. In particular, the methods are useful for treatment of autoimmune diseases, such as multiple sclerosis. Multiple sclerosis (MS), a human demyelinating disease which afflicts 600,000 individuals worldwide, results from damage of the myelin sheath of oligodendroglial cells in the Central Nervous System (CNS). Although the pathogenesis and etiology of MS have not yet been established, it is widely believed that the disease has an immunological basis and that both genetic and environmental factors make a contribution to the pathogenesis. The central mediator of autoimmune attack is believed to be host CD4+T cells specific for one or more autoantigens in the CNS, with subsequent production of an array of tissue-destructive inflammatory mediators following autoantigen-activation of these cells. Indeed, immunohistochemical analysis of the focal plaques of demyelination which occur in the brains of MS patients as a consequence of MS pathology have revealed the presence of CD4+T cells infiltrating these plaques.

Improved understanding of the immunopathological mechanisms underlying MS has developed from the study of experimental models of demyelination. The most commonly used model, experimental allergic encephalomyelitis (EAE), is an autoimmune inflammatory disorder of genetically susceptible mice that is mediated by autoantigen-specific CD4+MHC class II restricted T cells. In susceptible SJL/J mice, the disease can display a relapsing-remitting clinical course of paralysis, which makes it an ideal system to study the efficacy of various immunoregulatory strategies both in the prevention and treatment of disease.

The current invention is focused not on the cytokine-producing phenotype of autoreactive T cells in this disease setting, but on their trafficking from the host circulation to the site of pathology, for example within the CNS in the case of MS. As noted above, hemopoietic cell migration is regulated by chemokines.

In some embodiments, the invention provides antibodies directed against the extracellular domains of CXCR3, a 7-transmembrane chemokine receptor. Consistent with the chemotactic fingerprint of IP-10, CXCR3 is expressed exclusively on activated effector T lymphocytes. Exemplary peptides of the invention include peptides derived from the extracellular domains of the CXCR3 protein are presented in Table 1.

Peptides of the Present Invention

In preferred embodiments, the peptides of the invention are cyclized. Methods for cyclizing peptides are described in detail below. In those cases in which the peptides are cyclized by disulfide linkages, one of skill will recognize that the peptides will further comprise cysteine residues either within the peptide or at each terminus.

Polypeptides suitable for use in the present invention can be obtained in a variety of ways. Conveniently, they can be synthesized by conventional techniques employing automatic synthesizers, such as the Beckman, Applied Biosystems, or other commonly available peptide synthesizers using well known protocols. They can also be synthesized manually using techniques well known in the art. See, e.g. Stewart and Young, Solid Phase Peptide Synthesis, (Rockford, Ill., Pierce), 2d Ed. (1984).

Alternatively, DNA sequences which encode the particular chemokine receptor polypeptide may be cloned and expressed to provide the peptide. Nucleic acid molecules encoding chemokine receptors are known in the art and sequences of such genes are available, for instance, from GenBank (see, e.g., GenBank Accession Nos. HSU83326 HSU97123, AF005058). Nucleic acids encoding the CXCR3 human and mouse receptors are available as GenBank Accession Nos. X95876 and AF045146, respectively.

Standard techniques can be used to screen cDNA libraries to identify sequences encoding the desired sequences (see, Sambrook et al., Molecular Cloning—A Laboratory Manual, Cold Spring Harbor Laboratory, Cold Spring Harbor, N.Y., 1989). Fusion proteins (those consisting of all or part of the amino acid sequences of two or more proteins) can be recombinantly produced. In addition, using in vitro mutagenesis techniques, unrelated proteins can be mutated to comprise the appropriate sequences.

Chemokine receptor proteins from a variety of natural sources are also conveniently isolated using standard protein purification techniques. Peptides can be purified by any of a variety of known techniques, including, for example, reverse phase high-performance liquid chromatography (HPLC), ion-exchange or immunoaffinity chromatography, separation by size, or electrophoresis (See, generally, Scopes, R., Protein Purification, Springer-Verlag, N.Y. (1982)).

In some embodiments of the invention, the extracellular domain peptides are conformationally constrained. Means for achieving this are well known in the art (see, e.g., Hruby and Bonner in Methods in Molecular Biology, Volume 35: Peptide Synthesis Protocols Pennington and Dunn eds (Humana Press, Totowa N.J., 1994). A preferred means for preparing conformationally constrained peptides is through cyclization. Any method commonly used to produce cyclized oligopeptides can be used to produce the peptides of the invention. For example, in certain embodiments the peptides will include cysteine residues at both termini, which allow the production of cyclic peptides through disulfide linkages. Treatment of a such a peptide with an oxidizing agent such as oxygen, iodine or similar agent will produce a cyclic peptide which may be further purified using chromatographic or other methods of chemical purification. Construction of cyclic peptides can also be accomplished through thioether linkages. For instance, N-bromoacetyl-derivatized peptides can be reacted with sulfhydryl-containing residues, such as cysteine. Cyclization occurs by reaction of the free sulfhydryl of cysteine in the peptide with the bromoacetyl group to form a thioether linkage (Robey et al., Anal. Biochem. 177:373-7 (1989) and U.S. Pat. No. 5,066,716).

Other methods of constructing cyclic peptides are known to those skilled in the art. These include side chain-side chain, side chain-main chain and main chain-main chain cyclizations. In addition, linkers can be used to join the amino and carboxyl termini of a peptide. The linker is capable of forming covalent bonds to both the amino and carboxyl terminus. Suitable linkers are well known to those of skill in the art and include, but are not limited to, straight or branched-chain carbon linkers, heterocyclic carbon linkers, or peptide linkers. The linkers may be joined to the carboxyl and amino terminal amino acids through their side groups (e.g., through a disulfide linkage to cysteine) or through the alpha carbon amino and carboxyl groups of the terminal amino acids.

For a general discussion of suitable methods for cyclization see, Hruby and Bonner in Methods in Molecular Biology, Volume 35: Peptide Synthesis Protocols Pennington and Dunn eds (Humana Press, Totowa N.J., 1994). For instance, cyclizations may include formation of carba analogs and thioethers (Lebl et al. in Peptides 1986 Proceedings of the 19th European Peptide Symposium pp. 341-344; Robey et al., Anal. Biochem. 177:373-7 (1989) and U.S. Pat. No. 5,066,716), bis-thioethers (Mosberg et al. JACS 107:2986-2987 (1985)), azopeptides (Siemion et al. Mol. Cell. Biochem. 34: (1991)), and other cyclic structures, such as bridging structures (Charpentier, M., et al., J. Med. Chem. 32(6):1184-1190 (1989), Thaisrivongs, S., et al., J. Med. Chem. 34(4):127 (1991) and Ozeki, E., et al., Int. J. Peptide Protein Res. 34:111 (1989)). Cyclization from backbone-to-backbone positions may also be used.

Bridging is a special type of cyclization in which distant sites in a peptide are brought together using separate bridging molecules or fragments. Bridging molecules may include, for example, succinic anhydride molecules (Charpentier, B., et al., supra), and carboxymethylene fragments (Thaisrivongs, S., et al., supra). Bridging by metals can also be used (Ozeki, E., et al., supra).

In some embodiments, the peptides include two or more cystine residues. The cystines can be substituted or added within the peptide or at either terminus. The position of the cystines is not critical so long as disulfide linkages can form between them which allow the production of cyclic peptides. For example, treatment of such a peptide with an oxidizing agent such as oxygen, iodine or similar agent will produce a cyclic peptide which may be further purified using chromatographic or other methods of chemical purification.

Antibodies of the Present Invention

In addition to use of peptides, antibodies raised against peptides of the invention can be used to inhibit inflammatory responses. Antibodies can be raised to the peptides of the present invention using techniques well known to those of skill in the art. Anti-idiotypic antibodies can also be generated. The following discussion is presented as a general overview of the techniques available; however, one of skill will recognize that many variations upon the following methods are known.

A number of immunogens can be used to produce antibodies specifically reactive with the peptides. For instance, the entire chemokine receptor molecule or fragments containing the desired sequence can be used. Synthetic peptides as disclosed herein can be used either in linear form or cyclized.

Polyclonal antibodies

Methods of producing polyclonal antibodies are known to those of skill in the art. In brief, an immunogen (antigen), preferably a purified polypeptide, a polypeptide coupled to an appropriate carrier (e.g., GST, keyhole limpet hemanocyanin, etc.), or a polypeptide incorporated into an immunization vector such as a recombinant vaccinia virus (see, U.S. Pat. No. 4,722,848) is mixed with an adjuvant and animals are immunized with the mixture. The animal's immune response to the immunogen preparation is monitored by taking test bleeds and determining the titer of reactivity to the polypeptide of interest. When appropriately high titers of antibody to the immunogen are obtained, blood is collected from the animal and antisera are prepared. Further fractionation of the antisera to enrich for antibodies reactive to the polypeptide is performed where desired (see, e.g., Coligan (199 1) Current Protocols in Immunology Wiley/Greene, NY; and Harlow and Lane (1989) Antibodies: A Laboratory Manual Cold Spring Harbor Press, NY).

Monoclonal antibodies

In some instances, it is desirable to prepare monoclonal antibodies from various mammalian hosts, such as mice, rodents, primates, humans, etc. Description of techniques for preparing such monoclonal antibodies are found in, e.g., Stites et al. (eds.) Basic and Clinical Immunology (4th ed.) Lange Medical Publications, Los Altos, Calif., and references cited therein; Harlow and Lane, Supra; Goding (1986) Monoclonal Antibodies: Principles and Practice (2d ed.) Academic Press, New York, N.Y.; and Kohler and Milstein (1975) Nature 256: 495-497. Summarized briefly, this method proceeds by injecting an animal with an immunogen. The animal is then sacrificed and cells taken from its spleen, which are fused with myeloma cells. The result is a hybrid cell or “hybridoma” that is capable of reproducing in vitro. The population of hybridomas is then screened to isolate individual clones, each of which secrete a single antibody species to the immunogen. In this manner, the individual antibody species obtained are the products of immortalized and cloned single B cells from the immune animal generated in response to a specific site recognized on the immunogenic substance.

Alternative methods of immortalization include transformation with Epstein Barr Virus, oncogenes, or retroviruses, or other methods known in the art. Colonies arising from single immortalized cells are screened for production of antibodies of the desired specificity and affinity for the antigen, and yield of the monoclonal antibodies produced by such cells is enhanced by various techniques, including injection into the peritoneal cavity of a vertebrate (preferably mammalian) host. Specific monoclonal and polyclonal antibodies will usually bind with a K_{D} of at least about 0.1 mM, more usually at least about 50 μM, and most preferably at least about 1 μM or better.

Other suitable techniques involve selection of libraries of recombinant antibodies in phage or similar vectors (see, e.g., Huse et al. (1989) Science 246: 1275-1281; and Ward, et al. (1989) Nature 341: 544-546; and Vaughan et al. (1996) Nature Biotechnology, 14: 309-314).

Frequently, the peptides and antibodies of the invention will be labeled by joining, either covalently or non-covalently, a substance which provides for a detectable signal. A wide variety of labels and conjugation techniques are known and are reported extensively in both the scientific and patent literature. Suitable labels include radionucleotides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced. See, Cabilly, U.S. Pat. No. 4,816,567; and Queen et al. (1989) Proc. Nat'l Acad. Sci. USA 86: 10029-10033.

Humanized monoclonal antibodies

The antibodies of this invention can also be administered to an organism (e.g., a human patient) for therapeutic purposes (e.g., to inhibit an autoimmune response). Antibodies administered to an organism other than the species in which they are raised are often immunogenic. Thus, for example, murine antibodies administered to a human often induce an immunologic response against the antibody (e.g., the human anti-mouse antibody (HAMA) response) on multiple administrations. The immunogenic properties of the antibody are reduced by altering portions, or all, of the antibody into characteristically human sequences thereby producing chimeric or human antibodies, respectively.

Chimeric antibodies are immunoglobulin molecules comprising a human and non-human portion. More specifically, the antigen combining region (or variable region) of a humanized chimeric antibody is derived from a non-human source (e.g., murine) and the constant region of the chimeric antibody (which confers biological effector function to the immunoglobulin) is derived from a human source. The chimeric antibody should have the antigen binding specificity of the non-human antibody molecule and the effector function conferred by the human antibody molecule. A large number of methods of generating chimeric antibodies are well known to those of skill in the art (see, e.g. U.S. Pat. Nos: 5,502,167, 5,500,362, 5,491,088, 5,482,856, 5,472,693, 5,354,847, 5,292,867, 5,231,026, 5,204,244, 5,202,238, 5,169,939, 5,081,235, 5,075,431, and 4,975,369). An alternative approach is the generation of humanized antibodies by linking the CDR regions of non-human antibodies to human constant regions by recombinant DNA techniques. See Queen et al., Proc. Natl. Acad. Sci. USA 86:10029-10033 (1989) and WO 90/07861.

In one preferred embodiment, recombinant DNA vector is used to transfect a cell line that produces an antibody against a peptide of the invention. The novel recombinant DNA vector contains a “replacement gene” to replace all or a portion of the gene encoding the immunoglobulin constant region in the cell line (e.g., a replacement gene may encode all or a portion of a constant region of a human immunoglobulin, or a specific immunoglobulin class), and a “target sequence” which allows for targeted homologous recombination with immunoglobulin sequences within the antibody producing cell.

In another embodiment, a recombinant DNA vector is used to transfect a cell line that produces an antibody having a desired effector function, (e.g., a constant region of a human immunoglobulin) in which case, the replacement gene contained in the recombinant vector may encode all or a portion of a region of an antibody and the target sequence contained in the recombinant vector allows for homologous recombination and targeted gene modification within the antibody producing cell. In either embodiment, when only a portion of the variable or constant region is replaced, the resulting chimeric antibody may define the same antigen and/or have the same effector function yet be altered or improved so that the chimeric antibody may demonstrate a greater antigen specificity, greater affinity binding constant, increased effector function, or increased secretion and production by the transfected antibody producing cell line, etc.

Fully human antibodies

In another embodiment, this invention provides for fully human antibodies. Human antibodies consist entirely of characteristically human polypeptide sequences. The human antibodies of this invention can be produced in using a wide variety of methods (see, e.g., Larrick et al., U.S. Pat. No. 5,001,065). In one preferred embodiment, the human antibodies of the present invention are produced initially in trioma cells. Genes encoding the antibodies are then cloned and expressed in other cells, particularly, nonhuman mammalian cells. The general approach for producing human antibodies by trioma technology has been described by Ostberg et al. (1983), Hybridoma 2: 361-367, Ostberg, U.S. Pat. No. 4,634,664, and Engelman et al., U.S. Pat. No. 4,634,666. The antibody-producing cell lines obtained by this method are called triomas because they are descended from three cells; two human and one mouse. Triomas have been found to produce antibody more stably than ordinary hybridomas made from human cells.

Another method of providing fully human antibodies is described in Kucherlapati and Jakobovits, U.S. Pat. No. 5,939,598; Jakobovits et al. (1995) Ann. N.Y. Sci. Acad. Sci.: 764: 525-535; and Jakobovits (1995) Curr. Opin. Biotechnol. 6: 561-566. These antibodies have proved useful in the treatment of diseases and conditions. For example, a fully human monoclonal antibody to the human epidermal growth factor receptor produced using this technology can prevent the formation of human epidermoid carcinoma A431 xenografts in athymic mice (Yang et al. (1999) Cancer Res. 59: 1236-1243). These fully human antibodies are generated by injecting a mouse termed a XenoMouse™ (Abgenix, Inc., Fremont, Calif.) that has been engineered to generate an immunogenic response to an antigen to produce a fully human antibody. The mouse is produced by generating two separate transgenic mice which are bred to give rise to the XenoMouse™. Briefly, using gene-targeting technology one mouse is generated that has inactivated mouse Ig genes. This mouse is incapable of producing mouse antibodies. Another mouse is generated that contains YAC sequences encoding the human Ig heavy and κ light chains. This mouse strain is capable of producing human antibodies in response to an antigen, in the presence of mouse antibodies. These two mice are bred together to give rise to a mouse strain that can produce fully human antibodies in response to an antigen in the absence of mouse antibodies. These fully human antibodies can be produced in larger quantities using hybridoma or recombinant cell line technology. The peptides described in this invention can be used as antigens to challenge the XenoMouse™ in order to generate fully human antibodies to extracellular peptide sequences of the human CXCR3 receptor. These antibodies may prove useful in the treatment of disease or conditions related to inflammation involving the CXCR3 receptor.

Another method for producing fully human antibodies is described in U.S. Pat. Nos. 5,877,397; 5,874,299; 5,814,318; 5,789,650; 5,770,429; 5,661,016; 5,633,425; 5,625,125; 5,569,825; 5, and 545,806; and in Fishwild et al., (1996), Nature Biotechnol. 14: 845-851. These fully human antibodies can be generated by injecting a genetically engineered mouse known as a HuMAb-Mouse™ (Medarex, Inc.). Briefly, a mouse is genetically engineered using recombinant techniques to be homozygous for two different target deletions: a deletion of the endogenous heavy chain J segments, and a deletion of the endogenous κ-light-chain J and C segments. These deletions prevent the expression of the endogenous mouse IgM and Igκ. The mouse is also engineered to be hemizygous or homozygous for two transgenes—a germline, human, κ-light-chain transgene and a germline, human, heavy-chain, minilocus transgene. Such a mouse has been used to generate fully human antibodies against human CD4 (Fishwild et al., (1996)). Similar to the approach outlined with the XenoMouse™ above, the peptides of this invention can be administered to a HuMAb-Mouse™ to generate monoclonal antibodies against the CXCR3 receptor. Hybridoma technology can be used to generate larger quantities of the MAb of interest from an antigen-challenged mouse.

Formulation and Administration

The antibodies (typically monoclonal antibodies) of the present invention and pharmaceutical compositions thereof are useful for administration to mammals, particularly humans, to treat and/or prevent deleterious immune inflammatory responses, particularly those associated with autoimmune responses. Over 30 autoimmune diseases are presently known, including myasthenia gravis (MG), multiple sclerosis (MS), systemic lupus erythematosis (SLE), rheumatoid arthritis (RA), insulin-dependent diabetes mellitus (IDDM), and the like. Suitable formulations are found in Remington's Pharmaceutical Sciences, Mack Publishing Company, Philadelphia, Pa., 17th ed. (1985).

The antibodies of the invention are administered prophylactically or to an individual already suffering from the disease. The antibody compositions are administered to a patient in an amount sufficient to inhibit the recruitment of T cells to sites of inflammation. An amount adequate to accomplish this is defined as “therapeutically effective amount.” Amounts effective for this use will depend on, e.g., the antibody composition, the manner of administration, the stage and severity of the disease being treated, the weight and general state of health of the patient, and the judgment of the prescribing physician. The antibody compositions of this invention can be administered in amounts ranging from, but not limited to, 1-50 mg/kg, more preferably 5-20 mg/kg.

It must be kept in mind that the antibodies and compositions of the present invention may generally be employed in serious disease states, that is, life-threatening or potentially life threatening situations. For therapeutic use, administration should begin at the first sign of autoimmune disease. Antibody compositions can be administered prophylactically to a patient susceptible to or otherwise at risk of the disease to elicit an immune response against the target Chemokine receptor antigen.

The pharmaceutical compositions (containing antibodies) are intended for parenteral or oral administration. Preferably, the pharmaceutical compositions are administered parenterally, e.g., subcutaneously, intradermally, or intramuscularly. Thus, the invention provides compositions for parenteral administration, which comprise a solution of the immunogenic peptides dissolved or suspended in an acceptable carrier, preferably an aqueous carrier. A variety of aqueous carriers may be used, e.g., water, buffered water, 0.4% saline, 0.3% glycine, hyaluronic acid and the like. These compositions may be sterilized by conventional, well-known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as buffering agents, tonicity adjusting agents, wetting agents and the like, for example, sodium acetate, sodium lactate, sodium chloride, potassium chloride, calcium chloride, sorbitan monolaurate, triethanolamine oleate, etc.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%.

The antibodies of the invention can also be used for diagnostic purposes. For instance, antibodies can be used to detect the presence of particular Chemokine receptor molecules associated with disease.

The following examples are offered by way of illustration, not by way of limitation.

### EXAMPLE 1

This example describes a reproducible bioassay for CXCR3 activation.

A stable line transfectant of NSO-1 cells expressing the cDNA for human CXCR3 was prepared according to standard techniques. The surface expression of hu-CXCR3 on the transfectants compared to untransfected parent NSO-1 cells was confirmed by FACS staining using mouse anti-human CXCR3 monoclonal antibody (R&D systems).

To establish a bioassay for CXCR3 activation, the CXCR3 transfectant cells were cultured with either human IP-10, or the mouse anti-human CXCR3 antibody, and the physiological response of the cells was measured using a microphysiometer. This machine measures changes in the pH of the extracellular medium of the cell cultures which result from ligand receptor binding on the cell surface. These extracellular acidification rate measurements have previously been used as markers of antigen specific T cell activation and T cell epitope identification. The current assay is used to provide a biological read out for the identification of chemokine or chemokine receptor peptides involved in binding to IP-10 or agonistic anti-CXCR3 antibody.

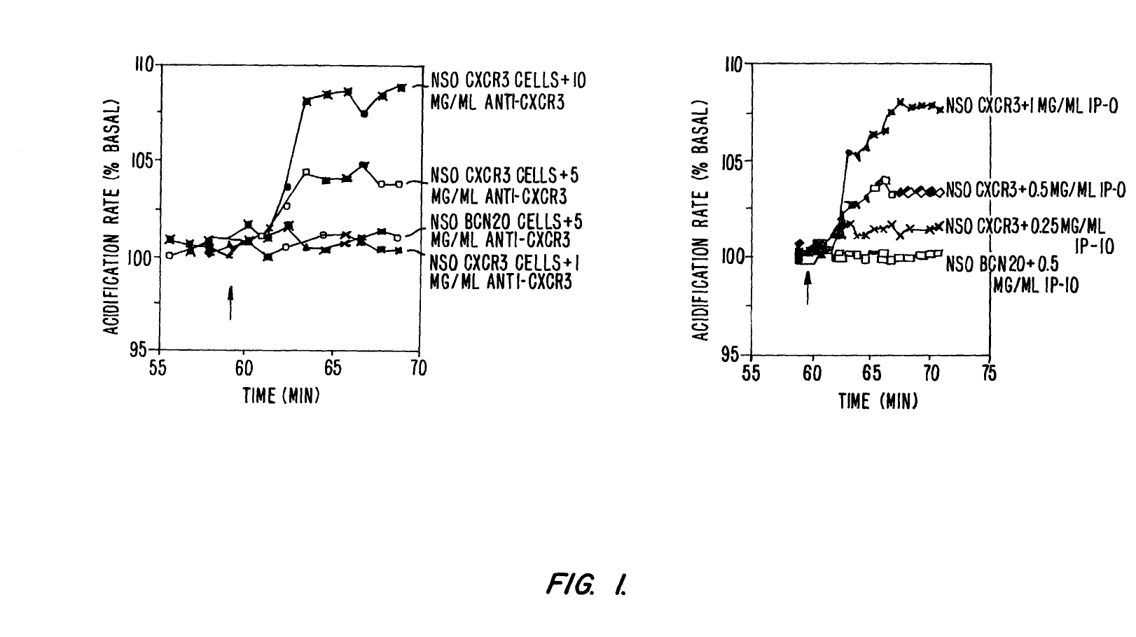

The experiments indicated that human IP-10 and anti-CXCR3 antibody both triggered substantial acidification rate changes in the CXCR3 transfectants, as shown in FIG. 1. Importantly, the same ligands induce no change in acidification rate of untransfected NSO-1 cells (FIG. 1).

### EXAMPLE 2

This example describes synthesis of human CXCR3 derived peptides.

A schematic representation of the surface portion of human CXCR3 is given in FIG. 2. The receptor has 4 surface portions, 4 intracellular portions and seven transmembrane portions. The surface portions were designated starting from N-terminus as SP-1, SP-2, SP-3 and SP-4.

The peptides shown in Table 1 derived from these four surface portions were prepared by solid phase peptide synthesis. The names of the peptides are based on the surface portion of CXCR3 from which they were derived. For example, SP-1-1 means this peptide was derived from the first portion of the CXCR3 protein.

TABLE 1

| Peptide | | | Amino acid |
|---|---|---|---|
| Name | Sequence | SEQ ID NO: | Position |
| SP-1-1 | MVLEVSDHQVLNDAEVAALL-NH₂ | 1 | 1-20 |
| SP-1-2 | ENFSSSYDYGENESDSCCTS-NH₂ | 2 | 21-40 |
| SP-1-3 | PPCPQDFSLNFDRAFLPA-NH₂ | 3 | 41-58 |
| SP-2-1 | DAAVQWVFGSGLCKV-NH₂ | 4 | 112-126 |
| SP-3-1 | SAHHDERLNATHCQYN-NH₂ | 5 | 191-206 |
| SP-3-2 | FPQVGRTALRVLQLVAG-NH₂ | 6 | 207-223 |
| SP-4-1 | DILMDLGALARNCGRESRVDVAKS-NH₂ | 7 | 278-301 |

The ability of these receptor-derived peptides to bind anti-CXCR3 antibody was evaluated using a standard ELISA format. The peptides were dissolved in 0.1 M bicarbonate buffer and coated on a 96 well ELISA plate overnight. The excess peptides were removed and nonspecific binding sites in the wells were blocked by 0.1 % bovine serum albumin. Anti-CXCR3 antibody (0.5 μg/well) was added to these wells and incubated for 2 hours. Excess antibody was removed by washing with PBS. HRP conjugated goat anti-mouse antibody was used as secondary antibody for detection. Two of the seven receptor-derived peptides, namely SP-1-3 and SP-4-1, showed substantial binding to anti-CXCR3 antibody. Further support for the conclusion that anti-CXCR3 antibody bound two of the seven receptor-derived peptides was provided by FACS analysis of the same interactions. These studies revealed that the receptor-derived peptide SP-4-1 potently blocked binding of anti-CXCR3 antibody to the CXCR3 cell line transfectant. The receptor-derived peptide SP-1-3 provided partial inhibition of the binding of the antibody to CXCR3 transfectants. In contrast, an antibody non-binding receptor-derived peptide, SP-2-1, failed to inhibit binding to CXCR3 transfectants. The data described here collectively demonstrate that anti-CXCR3 antibody can bind to two separate peptide portions of the CXCR3 extracellular domain.

### EXAMPLE 3

This example demonstrates that peptide vaccines of the invention can be used to prevent EAE in mice.

Protocols for Animal Experiments

SJL mice (6-8 weeks old) were obtained from Jackson Laboratories. They were kept in quarantine for two weeks. These mice get EAE when immunized with a peptide from proteolipid protein (PLP). The peptide sequence used for the immunization of these mice is PLP 139-151 and is amidated at the C terminus (HSLGWLGHPDKF-NH2). For the experiments, induction of the disease is considered Day 0. Three weeks before the induction of the disease, the mice were vaccinated with Human CXCR3 SP-4-1 peptide mixed with complete Freund's Adjuvant.

Preparation of Peptide CFA Emulsion for Treatment

4 mg of human CXCR3 SP-4-1 peptide was dissolved in 1 ml of phosphate buffered saline pH 7.4 (PBS). One ml of CFA obtained from VWR (Difco, Adjuvant Complete H37RA) was added to the peptide solution and the mixture was sonicated for 5 seconds using a fine tip sonicator. The emulsion was taken in 1 ml syringe (needed 2 syringes) fitted with a 25 gauge needle. Each mouse was given 100 μl of the emulsin under each flank near the hind legs by subcutaneous injection (total volume per mouse=200 μl, total peptide per mouse=200 μg).

Preparation of CFA Alone

One ml of PBS and 1 ml of CFA were mixed and sonicated for 5 seconds and the emulsin is drawn into a 1 ml syringe fitted with a 25 gauge needle. Each mouse was given 100 μl of this emulsion under each flank near hind legs by subcutaneous injection (total volume of CFA per mouse=200 μl).

Induction of Disease

To an aqueous solution of PLP 139-151 (4 mg per ml in PBS) equal volume of CFA was added and the mixture was sonicated for 5 seconds. The emulsion was drawn in a 1 ml syringe fitted with a 25 gauge needle and subcutaneously injected (200 μl total per mouse) in mice at the foot pad and back.

Testing the Antibody Response to SP-4-1 Peptide

The mice were bled at week 0 and week 5 and serum was tested by ELISA for the presence of antibodies against the human CXCR3 SP-4-1 peptide. Briefly, the SP-4-1 peptide dissolved in 0.1 M sodium bicarbonate buffer was plated on a 96-well ELISA plate overnight. The nonspecific binding sites on the plate were coated with 0.1% bovine serum albumin solution in PBS. The wells were washed and serum (diluted in PBS) was added to the wells and incubated at room temperature for 1.5 hours. The wells were then washed and HRP conjugated anti-mouse immunoglobulin antibody was used to detect the presence of anti-SP-4-1 antibodies). The results of this ELISA clearly indicate that the SP-4-1 treated mice show an antibody response against this peptide.

Results of EAE Study

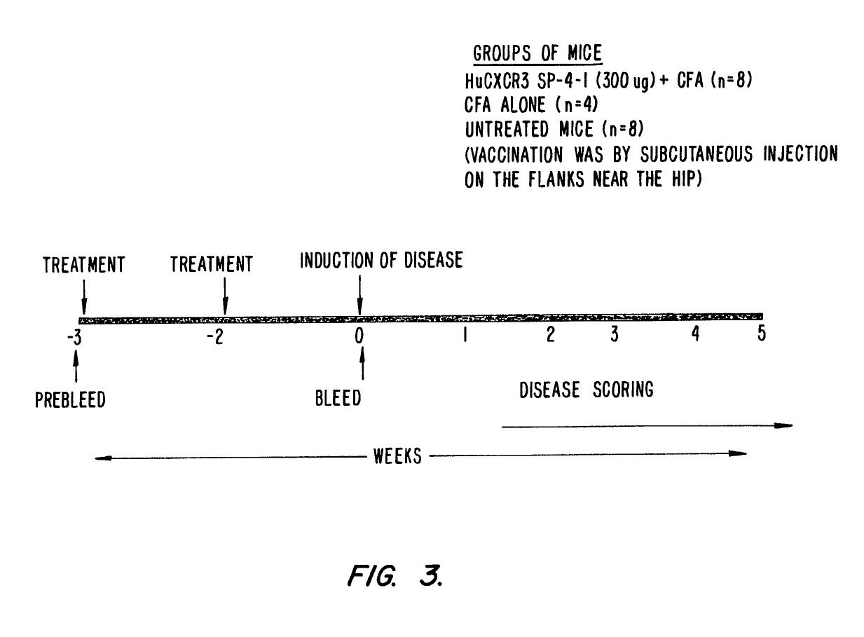

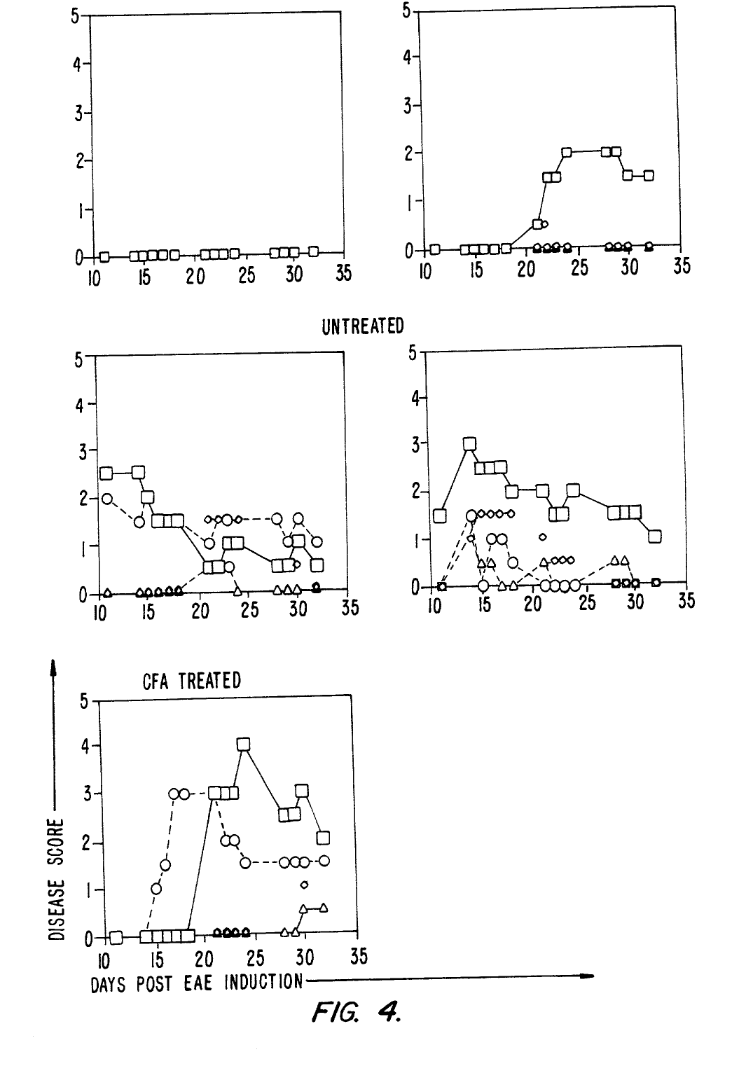

SP-4-1 was administered with CFA at 21 days and 14 days before induction of EAE as described above (FIG. 3). The results are shown in FIG. 4. There it can be seen that while most of the untreated and CFA treated mice showed clinical symptoms of EAE, 7 out of 8 mice treated with SP-4-1 in CFA showed no clinical symptoms.

### EXAMPLE 4

This example demonstrates that a monoclonal antibody to mouse CXCR3 1-37 can prevent EAE in SJL mice.

Monoclonal Antibody Production

A mouse CXCR3 1-37 peptide (MYLEVSERQVLDASDFAFLLENSTSPYDYGEN; SEQ ID NO: 8) was synthesized as peptide amide by Fmoc Chemistry using solid phase peptide synthesis. It was purified by reverse phase HPLC and characterized by mass spectrometry. The peptide was conjugated to PPD by using homobifunctional cross-linking agent BS3 (Pierce chemical Co). Rats were immunized with this conjugate and the lymph node or spleen cells were harvested on day 14 or day 60 respectively after immunization. The single cell suspension of spleen cells and lymph node cells from these rats were fused with myeloma SP2 cells using a Clone A cell kit (StemCell Technologies, Inc., Vancouver, British Columbia). The hybridoma colonies were grown in 96 well plate and the supernatants were tested for their ability to recognize the CXCR3 1-37 peptide by ELISA. Five clones from the lymph node preparations were found positive in the ELISA test. One of the clones, 1G3, was chosen for the production of pure monoclonal antibody. Pure monoclonal antibody (here after named as 1G3MAb) was obtained by Protein G affinity chromatography of 1G3 hybridoma cell culture supernatant. The antibody was also produced by the immunization of SCID mice with the 1G3 cells and purifying the 1G3mAb from ascites using Protein G affinity chromatography.

Animal Studies

SJL mice were divided into four treatment groups. EAE was induced in these mice by immunization with PLP 139-151 in CFA on day 0. The mice were subjected to either anti-CXCR3 1-37 Mab treatment or control treatments on day −1, +3 and +7. The control groups were given either isotype controlled Mab or PBS or no treatment. The mice were monitored for disease symptoms for 25 days after immunization. The results of the EAE experiment are summarized in Table 2.

TABLE 2
Prevention of EAE by anti-CXCR3 Mab treatment.

| | | | Mice exhibiting EAE/Total |
|---|---|---|---|
| | Treatment on Day | | number of mice in group |
| Group | +1, +3, +7 | Treatment Dose/route | on Day +25 |
| 1 | 1G3Mab | 200 μg/100 μl/injection | 1/7 |
| | (Rat anti-mouse | i.p | |
| | CXCR3 Mab) | | |
| 2 | Isotype ctr | 200 μg/100 μl/injection | 5/7 |
| | Rat IgG1 | i.p | |
| 3 | PBS | 100 μl i.p. | 10/20 |
| 4 | none | none | 14/20 |

The results clearly indicated that the anti-CXCR3 monoclonal antibody 1G3 prevents PLP peptide induced EAE in SJL mice. While 50-70 % of the mice in control group showed the symptoms of EAE, only 14 % of the mice treated with 1G3 Mab get the EAE indicating that the 1G3Mab is effective in the prevention of EAE.

### EXAMPLE 5

It is anticipated that fully human monoclonal antibodies could be generated by injecting a mouse comprising human immunoglobulin nucleic acid sequences, such as the XenoMouse™ (Abgenix, Inc.) or the HuMAb-Mouse™ (Medarex, Inc). Peptides such as SEQ ID NOS: 3,7, or 9 (MVLEVSDHQVLNDAEVAALLENFSSSYDYGENESDSC; CXCR3 1-37) could be introduced into the XenoMouse™ or the HuMAb-Mouse™ to generate monoclonal antibodies against extracellular regions of the human CXCR3 receptor. Monoclonal antibodies against the human CXCR3 extracellular regions would be promising candidates for the treatment of diseases involving inflammation, such as autoimmune diseases (e.g., multiple sclerosis).

The above examples are provided to illustrate the invention but not to limit its scope. Other variants of the invention will be readily apparent to one of ordinary skill in the art and are encompassed by the appended claims. All publications, patents, and patent applications cited herein are hereby incorporated by reference.

What is claimed is:

1. A method of inhibiting the recruitment of T cells to an inflammation site in a patient, the method comprising:
administering to said patient a therapeutically effective amount of a pharmaceutical composition comprising an antibody directed against an extracellular domain CXCR3 receptor polypeptide.

2. The method of claim 1, wherein said extracellular domain CXCR3 receptor polypeptide comprising a polypeptide from the first or fourth extracellular domain.

3. The method of claim 1, wherein said extracellular domain CXCR3 receptor polypeptide is SEQ ID NO: 3.

4. The method of claim 1, wherein said extracellular domain CXCR3 receptor polypeptide is SEQ ID NO: 7.

5. The method of claim 1, wherein said extracellular domain CXCR3 receptor polypeptide is SEQ ID NO: 9.

6. The method of claim 1, wherein said extracellular domain CXCR3 receptor polypeptide is cyclized.

7. The method of claim 1, wherein said antibody comprises a human antibody polypeptide sequence.

8. The method of claim 1, wherein said antibody consists of human antibody polypeptide sequences.

9. The method of claim 1, wherein the administration is parenteral.

10. The method of claim 1, wherein said patient is suffering from multiple sclerosis.

11. A pharmaceutical composition comprising:
a therapeutically effective amount of an antibody directed against an extracellular domain CXCR3 receptor polypeptide.

12. The pharmaceutical composition of claim 11, wherein said extracellular domain CXCR3 receptor polypeptide comprises a polypeptide from the first or fourth extracellular domain.

13. The pharmaceutical composition of claim 11, wherein said extracellular CXCR3 receptor polypeptide is SEQ ID NO: 3.

14. The pharmaceutical composition of claim 11, wherein said extracellular CXCR3 receptor polypeptide is SEQ ID NO: 7.

15. The pharmaceutical composition of claim 11, wherein said extracellular CXCR3 receptor polypeptide is SEQ ID NO: 9.

16. The pharmaceutical composition of claim 11, wherein said extracellular CXCR3 receptor polypeptide is cyclized.

17. The pharmaceutical composition of claim 11, wherein said antibody comprises a human antibody polypeptide sequence.

18. The pharmaceutical composition of claim 11, wherein said antibody consists of human antibody polypeptide sequences.
